(19) United States
(12) Patent Application Publication
(10) Pub. No.: US 2010/0061980 A1
(43) Pub. Date: Mar. 11, 2010

(54) ASSAYS TO PREDICT AND MONITOR ANTIBODY MEDIATED REJECTION OF TRANSPLANTED ALLOGRAFTS

(75) Inventor: Mieko Toyoda, Fullerton, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 12/516,524

(22) PCT Filed: Nov. 30, 2007
(86) PCT No.: PCT/US2007/024610

§ 371 Date: May 27, 2009

Related U.S. Application Data
(60) Provisional application No. 60/868,320, filed on Dec. 1, 2006.

(51) Int. Cl. A61K 39/395; G01N 33/00

(52) U.S. Cl. 424/130.1; 435/7.25

(57) ABSTRACT

The present invention is directed to assays that may be used to measure B cell reactivity to allo or donor antigens in patients, gauge the efficacy of desensitization treatment of these individuals, predict antibody mediated rejection, and monitor patients post transplant for antibody mediated rejection.

### STATEMENT OF GOVERNMENT RIGHTS

This invention has been developed pursuant to NIH/NIAID grant number U01 AI46134. The Government may have rights in this invention.

### FIELD OF THE INVENTION

The present invention is directed to assays useful for measuring B cell reactivity to allo or donor antigens (Ags) in patients having anti-human leukocyte antigen (HLA) and/or donor Ag-specific antibodies, gauging the efficacy of desensitization treatment of these individuals, predicting antibody mediated rejection (AMR) and monitoring patients post transplant for AMR and possibly chronic allograft rejection. In some embodiments, the assay uses flow cytometry to detect a patient's B cell reactivity to alto or donor Ags, which has been correlated by experimental results to allosensitization of patients and AMR.

### BACKGROUND

When a patient is in end stage organ failure, the most efficacious treatment option is usually a transplant operation where the diseased organ is replaced with a healthy organ from a deceased or live donor. The short term (one year) survival rate for patients receiving an organ transplant is relatively high, with the highest survival rate being over 96% for pancreas transplant procedures and a low of close to 60% for heart and lung transplants. The most significant risk to short term graft and patient survival is acute rejection of the transplanted organ. As can be seen from the high short term survival rates, however, acute rejection can often be effectively managed through improved tissue matching techniques, as well as improved immunosuppressant therapies.

Despite improvements prolonging the short term survival of organ transplant recipients, the long term graft survival rate is not optimal, having a high of around 80% for a pancreas transplant and low of around 40% for a heart and lung transplant after five (5) years. The graft survival rate drops further after ten (10) years, having a high of around 60% for liver transplants and a low of around 20% for heart-lung transplants. One of the significant factors leading to long term graft failure is chronic transplant rejection, which is characterized by fibrosis of the internal blood vessels of the transplant. It has been hypothesized that chronic rejection may be caused by sub-clinical AMR where B cells are consistently or periodically activated. However, this sub-clinical activation is generally not detectable by tests currently known in the art, and chronic transplant rejection remains untreatable.

Moreover, acute and chronic rejection is a particular problem for a certain subset of patients with high levels of anti-HLA antibodies (Highly Sensitized [HS] patients). Individuals can become Highly Sensitized through exposure to non-self HLAs from previous transplants, blood transfusions, and/or pregnancies. It is estimated that over 30% of the patients on the transplant waiting list are Highly Sensitized, and, as a result, will experience an increased number of rejection episodes and will have a lower rate of graft survival. Detection of these HS patients who have a higher risk for rejection episodes, however, remains problematic.

It is currently known in the art that patients having donor-specific antibodies show significantly worse graft outcomes than those without donor-specific antibodies. Despite this relationship, detection of donor specific antibodies using previously available methods does not always correlate well with clinical outcomes. For instance, one recent study detected donor specific antibodies in serum only in 42% of patients one (1) year post-transplant and 32% of patients at the time of graft failure. These results contrast with the detection of donor specific antibodies in 71% of the eluates from rejected kidneys, suggesting that the antibodies associated with graft failure are undetected in the circulating blood of many patients. Moreover, in a phenomenon called “accommodation,” some patients having donor specific antibodies do not develop AMR. Accordingly there remains a need in the art for a non-invasive test to detect HS patients who have a high risk for rejection.

Because HS patients represent a significant portion of the population needing an organ transplant, scientists and physicians have been developing therapies to modulate or suppress transplant recipient's humoral immunity. One of the most effective therapies is the administration of intravenous immunoglobulin (WIG) to HS patients prior to an organ transplant. This treatment reduces HS patients' allosensitization and increases transplant rate. IVIG is a blood product containing the pooled antibodies fractionated from the plasma of over 1,000 blood donors. The precise mechanism by which WIG suppresses a patient's immune response is unknown. One possibility is that some antibodies contained in WIG may bind directly with the abnormal host antibody, stimulating its removal. Alternatively, the massive quantity of antibody may absorb the host's complement components, preventing AMR. IVIG also interacts with various immune cells and suppresses their activation. In addition to providing effective desensitization for HS patients, IVIG therapy also improves long term allograft outcomes and is useful in treatment of acute allograft rejection episodes. However, the art currently lacks means to assess the efficacy of WIG desensitization treatment and determine which HS patients remain at risk for AMR.

Although HLA-sensitization has long been a problem in the transplant field, studies detecting HLA-specific B cells are limited. Initially, Mulder et al. (38) detected anti-HLA producing B cells in vitro in Peripheral Blood Mononuclear Cells (PBMCs) from multiparous women with serum anti-HLA antibodies using CD40L culture system, and measured their precursor frequencies. They found that anti-HLA antibodies produced from these cultured B cells showed similar specificity to those in serum and suggested the utility of the assay for HS patients awaiting transplant. In their follow up study (39), they developed a method for detection of HLA-A2-specific B cells isolated by HLA-A2 tetramers originally made for detecting Ag-specific T cells (29). However, they found that HLA-class I tetramers appeared unsuitable for detection and identification of allo-Ag-specific B cells in unsorted B cells obtained from HLA-immunized individuals.

Zachary et al. have reported the frequencies of HLA-specific B cells in HLA sensitized patients using HLA-A2, A24 and B7 tetramers loaded with HW peptides (40). They showed that the frequencies of tetramer+ B cells were significantly higher among sensitized patients including historic HLA antibody+patients than among non-sensitized patients, and cultured tetramer+ B cells produced antibodies specific for epitopes of the tetramer Ag and those cross reactive with the tetramer Ag as well. Their follow up study (41) showed that the frequency of tetramer+ B cells was correlated with tetramer specific antibody production after transplant among patients who were negative for tetramer specific antibodies at the time of transplant, suggesting the utility of tetramer staining for identifying patients who have documented or undocumented sensitization to assess the risk for AMR. However, the correlation between the tetramer+ B cell frequency and AMR episodes was not shown. In their study, the frequencies of tetramer+ B cells in HS patients were 4.1-5.5%. These were significantly higher than those among non-sensitized patients (1.6-3.2%) and normal controls (1.7%). In addition, the variation of the tetramer+ B cell frequency among their HS patients was fairly low (SD, 1.3-1.9%). In contrast, the medians of IFNγpositive (IFNγ+) B cell % in response to various PBMCs in pre-IVIG-Rx HS patient included in this study were fairly low (0.88% [range 0.03-7.9%] with donor PBMCs, 0.72 [0.01-7.8] with 3^{rd} N-ABOcom PBMCs and 0.79 [0.01-7.6] with 3^{rd} N-ABOincom PBMCs). In addition, the variation of IFNγ+ B cell % among patients was large as shown above.

The reasons for this discrepancy are uncertain. One possibility is that the two assays may detect different types of B cells in HS patients, although other possibilities such as different patient populations between 2 studies cannot be excluded.

### SUMMARY OF THE INVENTION

Among three of the major technologies to detect and/or quantify Ag-specific B cells and/or T-cells, HLA-multimer including tetramer, ELISPOT and cytokine Flow cytometry (CFC), the CFC method has several advantages as a diagnostic tool. It is not Major Histocompatability Complex (MHC)-restricted, unlike HLA-multimer (30), and can be done on whole blood, which is in contrast to ELISPOT that requires lymphocyte isolation from blood (34). Further, responding cells with different phenotypes can be separately analyzed by staining for surface markers which is impossible with ELISPOT (34). Quantification of cytokine secretion can also be achieved in this method, but not by ELISPOT.

The current invention seeks to address these problems by providing a novel assay to measure B cell reactivity to allo or donor Ag in HS patients treated with desensitization treatment prior to organ transplant procedures, which allows one to assess the efficacy of desensitization treatment of these patients pre-transplant and distinguish patients with a positive prognosis from patients who will likely experience AMR of the transplanted allograft and hence will require additional desensitization treatment before transplantation. Moreover, the inventive assay can also be used to monitor patients post-transplant to predict acute and/or chronic allograft rejections.

The present invention is directed to methods of detecting allo- or donor-specific B cells in a patient comprising: (a) treating a sample of blood from the patient with at least one co-stimulating antibody and a Golgi body secretion inhibitor to form a treated sample; (b) adding challenge antigens to the treated sample to form a challenged sample; and (c) analyzing the challenged sample and a control sample to detect IFNγ+ B cells; whereby an increase in the number of IFNγ+ B cells in a challenged sample as compared to a control sample is indicative of B cell activation. The terms “treatment” and “treating a sample” do not imply an chronological order when used to refer to the addition of multiple compositions. For example, the phrase “treating a sample with at least one co-stimulating antibody and a Golgi body secretion inhibitor to form a treated sample” does not specify the order in which these compositions are added to the sample. This means the Golgi body secretion inhibitor and co-stimulating antibody can be added in any order, either directly to a whole blood sample or to a portion thereof. One of skill in the art will appreciate that the addition of these compositions can also occur at any point in the methods herein, e.g., before or after the addition of the challenge antigen.

The present invention is also directed to methods of preventing long term graft rejection of a transplanted organ in a patient in need thereof, the method comprising: (a) treating a sample of blood from the patient with at least one co-stimulating antibody and a Golgi body secretion inhibitor to form a treated sample; (b) adding challenge antigens to the treated sample to form a challenged sample; (c) analyzing the challenged sample and a control sample to detect IFNγ+ B cells; whereby an increase in the number of IFNγ+ B cells in a challenged sample as compared to a control sample is indicative of B cell activation; and (d) correlating the presence of allo- or donor-specific B cells with a likelihood that the patient will undergo AMR or chronic rejection.

The present invention is also directed to diagnostic methods comprising (a) obtaining a sample of blood from a patient; (b) treating the sample of blood with at least one co-stimulating antibody and a Golgi body secretion inhibitor to form a treated sample; (c) adding challenge antigens to the treated sample to form a challenged sample; (d) analyzing the challenged sample and a control sample to detect IFNγ+ B cells; whereby an increase in the number of IFNγ+B cells in a challenged sample as compared to a control sample is indicative of B cell activation; and (e) providing a report of the analyzed results comprising the level of allo- or donor-specific B cells in the patient.

### BRIEF DESCRIPTION OF THE DRAWINGS

FIG. 1 illustrates that HS Patients show elevated antibodies in their blood prior to transplant (the dotted line represents normal levels), including elevated levels of anti-HLA antibodies (Class I and II), anti-endothelial cell antibodies (AECA), anti-cytomegalovirus (CMV) antibodies and total immunoglobulin G.

FIG. 2 illustrates an example picture of the B cell-CFC analysis of the present invention. Lymphocytes first gated by forward/side scatter graph (A) were further plotted against CD3 and CD8 (B). CD3+/CD8+, CD3+/CD8−, CD3−/CD8^{dim+} and CD3−/CD8− were tentatively designated as T8, T4, NK and B cells, and IFNγ+ cell % in each cell population was calculated (C-F).

FIG. 3 illustrates IFNγ+ cell % in B, NK, T8 and T4 cells pre-IVIG-treatment (Rx) in 15 HS patients and 14 normal individuals (3^{rd} N) without PBMC stimulation. IFNγ+ cell % in HS patients were tested pre-IVIG-Rx (one time point). IFNγ+ cell % in 3^{rd} N were the average of IFNγ+ cell % tested at multiple time points in one individual (5.4±5.6 time points/individual, range 1-21). A dot and vertical bar represent mean and SD, and a short horizontal bar represents median.

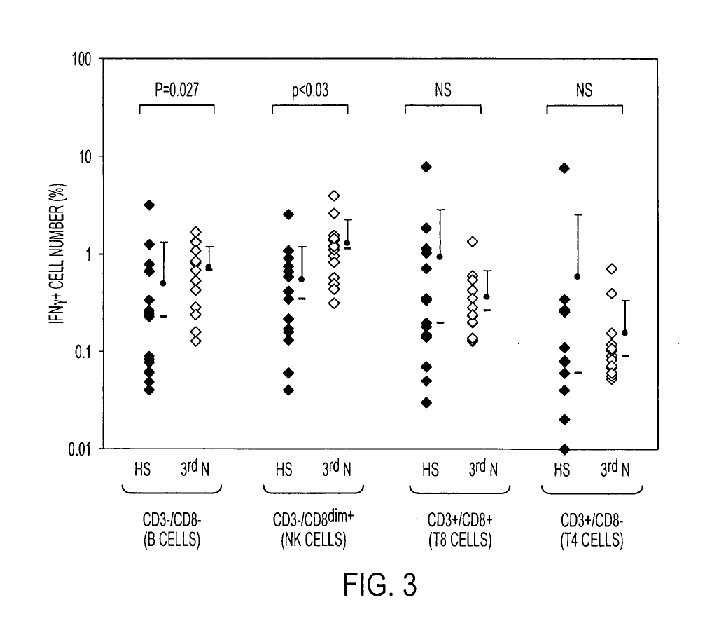

FIG. 4 illustrates an association between IFNγ+ B cell % and anti-HLA class I or class II Ab levels pre-IVIG-Rx in 15 HS patients and 14 normal individuals (3 N) without PBMC stimulation. IFNγ+ cell % and anti-HLA Ab levels in HS patients were tested pre-IVIG-Rx (one time point). IFNγ+ cell % and anti-HLA Ab levels in 3^{rd} N were the average of IFNγ+ cell % and anti-HLA Ab levels tested at multiple time points in one individual (5.4±5.6 time points/individual, range 1-21 for IFNγ+ cell %; 2.5±1.4 time points/individual, range 1-6 for anti-HLA Ab levels).

FIG. 5 illustrates a rate of IFNγ+ B cell % increase in response to various PBMCs pre-IVIG-Rx in 15 HS patients and 14 normal individuals (3^{rd} N). The results are expressed as the ratio against IFNγ+ B cell % without PBMC stimulation in each blood sample. A ratio >1.0 represents positive response (the dotted line represents a ratio of 1.0). Rate of IFNγ+ B cell % increase in HS patients were tested pre-IVIG-Rx (one time point). HS patient blood samples were stimulated with PBMCs obtained from donor (ABOcom), 3^{rd} N-ABOcom or 3^{rd} N-ABOincom as shown in Table 2. 3^{rd} N-ABOcom and ABOincom PBMCs were obtained from multiple donors (3^{rd} N-ABOcom PBMCs: A-1, B-1, AB-1, O-3; 3^{rd} N-ABOincom PBMCs: A-4, B-2, AB-0, O-2). Rates of IFNγ+ B cell % increase in 3^{rd} N were the average of IFNγ+ B cell % increase rates tested at multiple time points in one individual (5.4±5.6 time points/individual, range 1-21). 3^{rd} N blood samples were stimulated with PBMCs obtained from HS (ABOcom), donor (ABOcom), or 3^{rd}-ABOincom. 3^{rd} N-ABOincom PBMCs were obtained from multiple donors (2.4±1.6 donors/3^{rd} N, range 1-6). A dot and vertical bar represent mean and SD, and a short horizontal bar represents median.

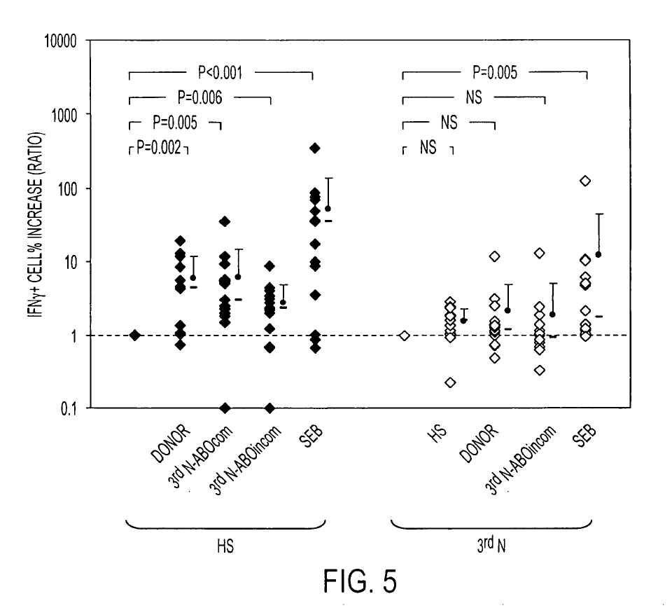

FIG. 6 illustrates an association between rate of IFNγ+ B cell % increase in response to various PBMCs and anti-HLA class I or class II Ab levels in 15 pre-IVIG-Rx HS patients. Rate of IFNγ+ cell % increase and anti-HLA Ab levels in HS patients were those tested pre-IVIG-Rx (one time point).

FIG. 7 illustrates a rate of IFNγ+ B cell % increase in response to various PBMCs before, during and after IVIG-Rx, and after transplant in HS patients who received a transplant. The results are expressed as the ratio against FNγ+ B cell % without PBMC stimulation in each blood sample. A ratio >1.0 represents positive response. A vertical dotted line and open arrows represent transplant date and IVIG-Rx, respectively. Patient #s correspond to those in Table 2.

### DETAILED DESCRIPTION

As used in this specification and the accompanying figures, the notation “+” is used to indicate “positive.” For example, “IFNγ+” can be read as “IFNγpositive.” The notations Rx and Tx are abbreviations for “treatment” and “transplant,” respectively.

The current invention provides a novel assay to measure B cell reactivity to allo- or donor-Ags in patients in need thereof and methods of using this assay. In some embodiments, this assay is used in HS patients. The term Highly Sensitized refers to those patients who are sensitized against allo-Ags including HLA and/or non-HLA antigens. HS patients show positivity for antibodies to allo-Ags (primarily HLA antigens) in their blood as detected by various assays such as a complement dependent cytotoxicity assay, luminex assay and flow cytometry. In contrast, normal individuals, i.e., non-Sensitized individuals, usually show negative for these assays.

In some embodiments, the patient from whom a sample is taken for this assay has been treated with desensitization treatment, e.g. IVIG, prior to organ transplant procedures. Suitable desensitization treatments include WIG, anti-CD20 antibody, and/or plasmapheresis. A suitable dose of IVIG is usually 2 g/kg. As one of skill in the art will appreciate, however, this dose can be varied depending on the patient's condition. For example the IVIG dose can range from about 0.5 kg/g, 1 g/kg, or 1.5 g/kg up to about 2 g/kg. In some embodiments, the preferred dose is about 1-2 g/kg or about 2 g/kg.

In some embodiments, a combination of WIG and anti-CD20 antibodies can be used as the desensitization treatment. In some instances, this combination has shown a higher efficacy for desensitization compared to WIG alone. In other embodiments, e.g., in those patients who still show high levels of anti-HLA antibodies after IVIG procedures, plasmapheresis can be used as the desensitization treatment.

In some embodiments, the inventive assay can be used to assess the efficacy of the desensitization treatment of these patients pre-transplant and distinguish patients with a positive prognosis from patients who will likely experience AMR of the transplanted allograft and hence will require additional desensitization treatment before transplantation. Moreover, the inventive assay can also be used to monitor patients post-transplant to predict acute and chronic allograft rejections.

Some embodiments of the invention are directed to taking blood samples from HS patients to be tested with the inventive CFC assay. Whole blood samples from normal individuals can be tested as a control group (Third Party (1)) while blood samples from these same individuals can be used for preparation of PBMCs serving as challenge Ags. PBMCs isolated from donors and another group of third party individuals (Third Party (2)) can also be used challenge Ags.

Data concerning the ABO blood types of the Third Party normal individuals can be analyzed to see whether ABO mismatch plays any role in positive results in the assay. The data are analyzed as ratios of the IFNγ+ B cells in stimulated samples (i.e., with third Party or Donor PBMCs) to those in control (unstimulated) matching samples. As described more below, a ratio greater than 1 indicates a positive response.

Moreover, the inventive assay can also be used to predict AMR, and gauge the efficacy of desensitization treatments. Patients with positive B cell-CFC, especially against donor Ags are likely to develop AMR and may need additional desensitization treatments prior to transplantation. Monitoring Donor-specific B cell responses by the B cell-CFC during and after desensitization protocol may increase AMR free transplantation. Monitoring patients post-transplant by the B cell-CFC may predict AMR or chronic rejection.

The following sections detail some embodiments of the inventive assay and methods of using it in more detail. The headings are provided for organizational purposes only and are not intended to impart any meaning or division to this document unless specified herein.

B Cell Activation is an Indication of Antibody Mediated Rejection (AMR) and of HS Patients

B cells play a primary role in a patient's humoral immunity. These cells are responsible for the production and maintenance of antibodies and are present among the cells that infiltrate rejected organs. Accordingly, one object of the inventive assay is to detect allo- and donor-Ag-specific B cells, which provides an indication of a patient's humoral immunity. Specifically, the inventive assay measures B cell reactivity against allo- and donor-Ags to assess the real time status of a patient's humoral immunity, to assess the efficacy of treatments to modulate the patient's humoral immunity, and to give important prognostic post transplant information.

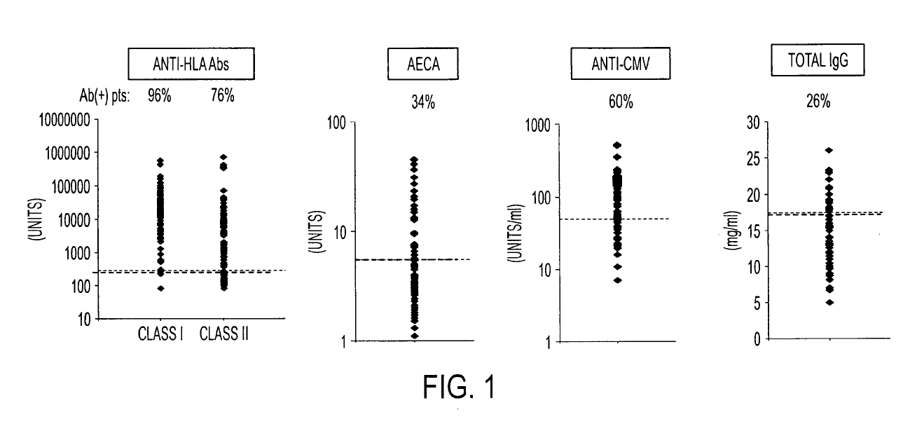

As illustrated in FIG. 1, HS patients show elevated antibodies in their blood prior to transplant (the dotted line represents normal levels), including elevated levels of anti-HLA antibodies (Class I and II), anti-endothelial cell antibodies (AECA), anti-cytomegalovirus (CMV) antibodies and total immunoglobulin G (IgG). It has been observed that for anti-HLA class I and class II 96% and 76% of a total of 83 HS patients tested showed higher levels as compared to the normal levels, respectively. The levels of other antibodies, AECA, anti-CMV and total IgG were also elevated in many patients: These elevated antibody levels suggest polyclonal B cell activation.

Moreover, as shown in Table 1, patients with lower levels of these antibodies pre- or post WIG treatment have a lower risk for AMR of the transplanted allograft. In the table AR is used rather than AMR to denote antibody mediated rejection, and PRA stands for panel reactive antibodies.

TABLE 1
Antibody levels pre- and post-IVIG treatment
(Rx) in patients who developed AMR vs. no AMR.

| | Antibodies | w/o AR (n = 7) | w/AR (n = 10) | p |
|---|---|---|---|---|
| Pre-Rx | Anti-HLA Class I (units) | 6,234 ± 5,416 | 31,652 ± 42,198 | N.S. (0.09) |
| | Anti-HLA Class II (units) | 232 ± 164 | 9,882 ± 9,178 | 0.009* |
| | PRA (%) | 52.3 ± 32.7 | 66.7 ± 27.7 | N.S. |
| | PRA w/DTT (%) | 52.6 ± 26.9 | 63.4 ± 32.8 | N.S. |
| Post-Rx | Anti-HLA Class I (units) | 4,798 ± 3,894 | 15,953 ± 19,682 | N.S. (0.11) |
| | Anti-HLA Class II (units) | 197 ± 103 | 7,071 ± 7,760 | 0.021* |
| | PRA (%) | 47.7 ± 30.5 | 48.9 ± 32.4 | N.S. |
| | PRA w/DTT (%) | 45.7 ± 20.5 | 55.9 ± 33.9 | N.S. |

*p < 0.05: with vs. without AR episodes.

These experimental results show that abatement of B cell activation is an essential element for successful transplant procedures in HS patients. Accordingly, detection of B cell reactivity can be used to detect HS patients who are highly reactive with allo- and/or donor-Ags and gauge the efficacy of desensitization treatments pre-transplant, and predict AMR and evaluate its corresponding treatment pre- and post-transplant. Serum samples were also treated with dithiothreitol (DTT) to delete anti-HLA IgM antibody.

Detection of B Cell Reactivity in Patients

Despite the link between B cell activation, allosensitization and AMR, studies regarding the detection of HLA-specific B cells are limited. The current invention overcomes this limitation by providing an assay to detect B cell reactivity against allo- and/or donor-Ags in a patient. In some embodiments, the assay uses intracellular cytokine flow cytometry (CFC) to measure a patient's B cell response to allo- or donor-specific Ags. The use of CFC is advantageous in these inventive assays because it can distinguish IFNγ+ cells separately in CD4, CD3, CD20, and other cell types. However, as one of skill in the art will appreciate, alternate assays with the ability to detect IFNγ+ cells by cell type can also be used to detect allo- and donor-Ag-specific B cells in the inventive assays described herein.

CFC is particularly advantageous because it is not restricted to the Major Histocompatibility Complex (MHC), and can be performed on samples of a patient's whole blood. In addition, CFC can separately analyze responding cells with different phenotypes by staining for surface markers. Moreover, CFC detects higher frequencies of antigen-specific cells, and CFC can quantify cytokine secretion.

In some embodiments, the inventive CFC assay detects Ag-specific B cells by measuring intracellular cytokine production by a patient's B cells in response to challenge Ags, and quantifies the frequency of antigen-specific cell responses. In one embodiment, the measured cytokine is IFNγ, a type II interferon, and the challenge Ags are PBMCs obtained from an organ donor or third parties (single or pooled multiple donors). As one of skill in the art will appreciate, other cytokines can be measured using this assay depending on the challenge antigen used and/or the target of the assay. For example, other cytokines involved in B cell activation or other molecules that are involved in B cell activation (e.g., inducible membrane molecules such as the adhesion molecule ICAM-1) may be used to detect B cell activation concurrent with IFNγdetection or independent of IFNγ detection.

The PBMCs used in the inventive assay can be from the donor, another person, or can be a mixture of PBMCs from multiple persons. Experiments show that PBMCs from a single donor or mixed PBMCs from multiple donors show no, or minimal, production of the cytokine IFNγ as detected by CFC. Consequently, both single and mixed PBMCs can be used as challenge Ags for the inventive CFC assay without interfering with the measurement of the patient's B cell activation. Although donor PBMCs are ideal for measuring the B cell reactivity of a patient in a transplant setting, measuring the global B cell reactivity against a mixture of PBMCs prepared from multiple normal individuals can also be informative. In some transplant situations there may be insufficient time to carry out the tests once the donor organ is identified. Therefore, it is useful to make an earlier assessment using the mixed donor PBMCs. Additionally, B cell reactivity against a mixture of third party PBMCs can be utilized for patients receiving deceased allografts where donor blood is difficult to obtain. In most cases the challenge PBMCs used should be irradiated to make certain that they are incapable of producing IFNγ.

The inventive assays can also use whole blood as the challenge Ag but in these embodiments the blood sample should be ABO matched to the whole blood challenge Ag to prevent clotting.

As one of skill in the art will appreciate, instead of PBMCs and whole blood, alloantigens or any antigens of interest can be used as the challenge Ags. Natural or recombinant HLA antigens are now available in a number of different forms including pure antigens and antigens bound to the surfaces of micro-beads. In this case, the benefit of purified antigens is that one can measure B cell reactivity against known antigens. However, this approach may miss B cell reactivity against antigens expressed on PBMCs other than HLA or similar known antigens and it is likely that those other antigens may be important causes of rejection.

One can also use other cell types such as endothelial cells, epithelial cells, etc. in the present invention. In particular, B cell reactivity against endothelial cells may be very important since these cells are the primary target on transplanted organs.

Preparation of Samples for Flow Cytometry

In some embodiments, whole blood samples from the patient to be tested are prepared for CFC. PBMCs are isolated from the organ donor or third parties, such as approximately five to ten (5-10) healthy adults, by density-gradient centrifugation using Ficoll-Hypaque gradients, or other techniques known in the art. The donor PBMCs are then aliquoted and frozen. If PBMCs are pooled from a plurality of third parties, the PBMCs are individually isolated from the individual blood samples, mixed, and then frozen. Blood is then drawn from the patient to be tested using standard techniques, and mixed with an anti-coagulant such as sodium heparin. Frozen test PMBCs are thawed and irradiated right before use. The whole blood samples can then be incubated with the irradiated test PBMCs (donor or pooled) in the presence of a Golgi inhibitor as Brefeldin A. Because such substances inhibit Golgi mediated secretion an accumulation of cytokines results within the patient cells.

In one embodiment, the test blood samples are divided into a plurality of groups, including control and experimental groups. The typical assay analyzes five different samples for each patient by flow cytometry: a positive control, a negative control, one or two stimulated experimental patient samples and a background control. A sample (1 ml) of patient whole blood is treated with co-stimulating monoclonal antibodies (10 microliters of anti-CD28 and anti-CD49d) and 10 microliters of Brefeldin A to inhibit Golgi secretion. Approximately two hundred (200) microliters of this mixture are aliquoted into four of the sample tubes. Experimental tubes are created by adding two hundred (200) microliters of donor PBMCs to a first tube and two hundred (200) microliters of third party PBMC mixture to a second experimental tube. The donor PBMCs and/or third party PBMC mixture should have a concentration of approximately 5×10⁶ cells per milliliter. The positive control is created by adding two (2) microliters of Staphylococcal Enterotoxin B (SEB, 1 μg/ml) to the third tube containing the patient's aliquoted blood. Mitogens and ionomycin can also be used as polyclonal stimulator instead of SEB. The negative control is the fourth tube which contains the aliquoted experimental patient's blood with no further additives. The background tube contains two hundred (200) microliters of the third party PBMC mixture. In some cases where the donor PMBCs are not available only the mixed third party PBMCs are used and there is only one experimental tube in the assay.

After the experimental tubes are made, all the tubes are incubated for approximately six (6) hours at a temperature close to body temperature (about 37° C.). In one embodiment, the six hours of incubation is followed by incubation at 18° C. overnight using a thermocycler. Prior to analysis the red cells in the samples are lysed in a lysis buffer (such as ammonium chloride or ammonium oxalate or proprietary mixtures such as FACS lysing solution) as is well know in the art. The unlysed cells remaining in the tubes are then fixed, using techniques known in the art, and permeabilized to facilitate intracellular staining with anti-IFNγantibodies. In one embodiment, the cells remaining in the test tubes are washed with buffer (0.5% Bovine Serum Albumin (BSA) in phosphate buffered saline (PBS) with 0.1% sodium azide as a preservative) and resuspended with a permeabilizing solution such as FACS Permeabilizing Solution 2. The presence of anti-IFNγantibodies within the cells indicates the production of the IFNγ by activated cells.

In addition, cell surface markers can be stained with monoclonal antibodies to identify different types of leucocytes. A preferred staining cocktail can contain fluorescently labeled anti-CD19, anti-CD4, anti-CD8 and anti-CD16 antibodies to differentiate B cells from T-cells and NK cells. The anti-CD4 and anti-CD8 antibodies bind to surface antigens on T-Cells and the anti-CD16 antibodies to surface antigens on NK cells while the anti-CD19 antibodies bind to surface antigens of B cells. As is well known in the art of flow cytometry it is possible to use different fluorescent labels for each of the antibodies so that they can be identified according to the wavelength of their fluorescence. Alternative monoclonal antibodies can readily be used for B cell and T cell detection as is well known in the art. For example, anti-CD20 can also be used to detect B cells instead of anti-CD19. For the detection of total T cells, anti-CD3 antibody should be used since CD4 and CD8 are two primary subsets of CD3 positive cells.

The cocktail of monoclonal antibodies can be varied depending on the experimental conditions and the treatment the patient has received prior to this assay. For example, if the patient has been treated with anti-CD20 antibodies the treatment depletes the patient's B cell count. This can effect the use of anti-CD20 or anti-CD19 antibodies to detect B cells. Thus, the cocktail used in the inventive assay could be varied to detect the CD3− population, which includes B cells.

As one of skill in the art will appreciate, this protocol for preparing samples may be varied without departing from the scope of this invention. Further methods are provided in the Examples below.

Flow Cytometry of the Prepared Samples

Once the cells are prepared, for example, according to the steps described above or in the Examples below, they are ready for analysis by means of flow cytometry. The samples are analyzed with a flow cytometer, such as those manufactured by BD Biosciences, Beckman Coulter or Dako, according to standard methods known in the art. During flow cytometric analysis the sample cells are hydrodynamically focused into a single file of cells which then pass one at a time through a light beam (usually laser). The light beam excites fluorescence in any antibodies attached to the cells which is detected by fluorescence sensors while light scatter sensors detect the presence and size of cells independent of any fluorescent antibody. In one embodiment, lymphocyte subset analysis can be performed using a standard staining procedure for flow cytometry. This analysis can be useful when interpreting CFC assay results since most patients receive T- and B cell depleting agents such as Campath 1H, Thymoglobulin and Rituximab.

From the flow cytometry analysis, the IFNγ+ cell frequency in CD19+, CD4+, CD8+ and CD16+ cell population can be determined. To obtain these results it may be necessary to use software accompanying the flow cytometer, such as Dako's Summit software. In one embodiment, the result of these analyses can be expressed as the percentage of IFNγ+ cells in each cell population. In an alternate embodiment, the results may be expressed as the ratio of a test patient's IFNγ+ cells to the IFNγ+ cells of an un-stimulated sample.

Results of Flow Cytometric Analysis

As previously mentioned, B cell activation in a patient correlates with that patient's allosensitization. Accordingly, the results of the inventive flow cytometric assay can be used by a physician, scientist or clinician to detect HS patients who have high reactivity to HLA and/or donor Ags and gauge the efficacy of desensitization treatments. This diagnostic capability has been confirmed experimentally, as described here and in the Examples below.

The ratio of IFNg+ cell % against that without stimulation can be used to determine if a positive response, i.e., B cell activation, was observed in the assay. A ratio >1.0 represents positive response in some embodiments. A ratio >5.0 is considered as a highly positive response since patients with AMR usually showed >5.0.

The impact of these ratios on the likelihood of developing AMR can depend on the characteristics of the patient being tested and should be considered. For example, the ratio in female patients, especially those who have been pregnant, is significantly higher than male or female without pregnancy. The ratio in females with AMR was significantly higher than those without AMR. However, the ratio in females without AMR was significantly higher than that in males without AMR and was similar to that in males who could not get a transplant. Thus, the cutoff levels for determining whether a positive response indicates AMR can differ from males to females and should be normalized accordingly.

In some embodiments, if the ratio is <5, it indicates that B cells have been activated but not to a level that will lead to AMR. In some embodiments, if the ratio is between 5 and 10, men have high risk for AMR, but women have about a 50:50 risk of AMR. If the ratio is >10, the patient has a very high risk for AMR regardless of gender.

A physician can use these ratios to determine which patients are at risk for AMR and designate them for additional desensitization or other treatments. Those with a low risk of AMR, for example those patients with a ratio of less than 5, can be identified for an organ transplant. Those with unacceptable risk levels, e.g., a ratio of over 5, can receive further treatment before receiving a transplant.

Attempts to detect Ag-specific memory B cells and investigate their function in patients with autoimmune diseases have been reported (33-35), since memory B cells play an important role in long-term humoral immunity (32) and reside in secondary lymphoid organs and the periphery. Thus, detecting allo- and/or donor-specific B cells and assessing their immunity is relevant to the prediction of AMR in HS patients.

As described below in the Examples and as shown in the Figures, experiments showed elevated levels of various HLA and non-anti-HLA antibodies in HS patients, indicating polyclonal B cell activation. Patients who showed significant reduction of these antibodies during and after IVIG-Rx showed higher transplant rates compared to those who did not, and those with lower levels of these antibodies pre- or post-IVIG-Rx had lower risk for AMR (unpublished data). While not wishing to be bound by a single theory, it is believed that abatement of B cell activation is essential for successful transplant in this patient population. Thus, detection of activated B cells can gauge the degree of sensitization, IVIG-Rx efficacy and predict AMR risk post-transplant.

Since memory B cells can be activated directly by cognate non-processed antigens through surface Ig, which results in the production of cytokines such as IFNγ(36), the inventive B cell-CFC assay has been developed to detect activation of allo- and donor-Ag specific B cells. In this assay, whole blood (responder) is incubated with donor or 3^{rd} N PBMCs as allo- and donor-Ags (stimulator), and IFNγ+ B cells are enumerated by flow cytometry. It was examined whether IFNγ+ B cell % in pre-IVIG-Rx HS patient blood without stimulation was elevated comparing to 3^{rd} N. Unexpectedly, those in HS patients were even lower than those in 3^{rd} N although the levels were largely overlapped between the 2 groups. The same was true for NK cells, but not T cells which showed similar levels in both HS patients and 3^{rd} N. It should be noted that HS patients showed more variance in IFNγ+ cell % in all cell populations comparing to that in 3^{rd} N. This might reflect the greater variance in the immunological state in the HS patient population. However, IFNγ+ B cell % was not associated with anti-HLA antibody levels, suggesting that both factors are independent.

It was also examined whether IFNγ+ B cells increased when blood was stimulated with various PBMCs. IFNγ+ B cells responding to donor, 3^{rd} N-ABO compatible or incompatible PBMCs were significantly elevated comparing to those without stimulation in many HS patients, while B cell response to HS patient, donor or 3^{rd} N-ABO incompatible PBMCs did not change in most normal individuals. These results suggest that allo- and/or donor Ag-specific memory B cell numbers are elevated in many HS patients, but not in most normal individuals, and the B cell responses seen in HS patients are not against ABO Ags. B cell response against SEB was also significantly higher in HS patients comparing to normal individuals. This is likely due to the elevated number of activated memory B cells in HS patients and those being activated by cytokines produced by or CD40L expressed on SEB-activated T cells (37).

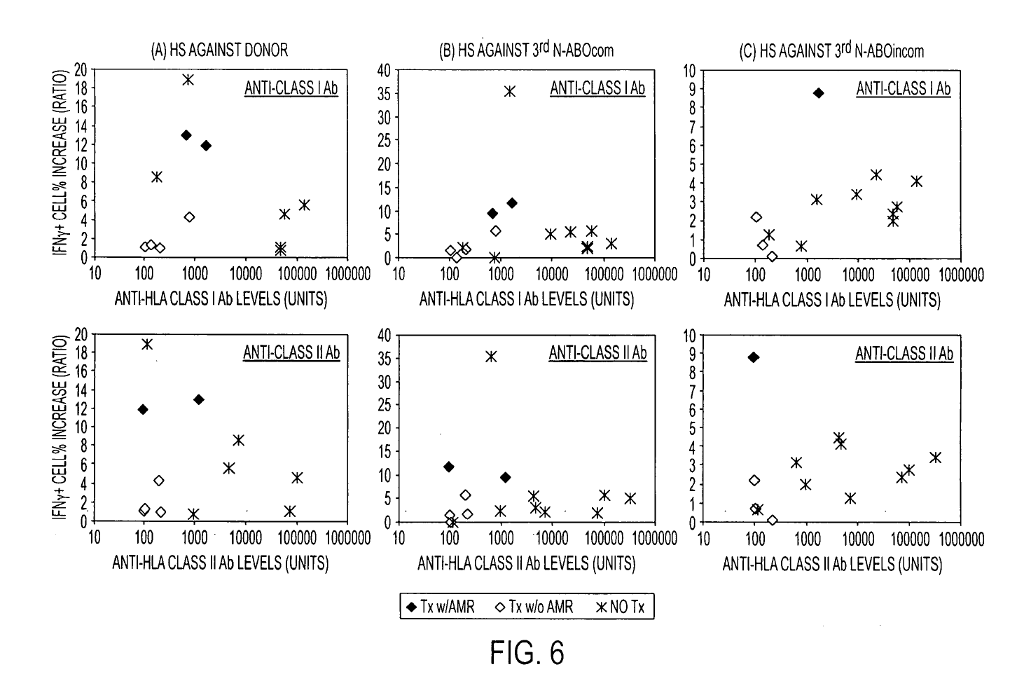

Although elimination of anti-HLA antibodies and monitoring patients for these antibodies during and after desensitization using sensitive and specific assays are essential for successful transplantation in HS patients, antibodies detected by these tests do not always correlate well with clinical outcomes (23, 24). Currently, the minimal antibody criteria for transplant at Cedars-Sinai Medical Center is a negative CDC CMX and flow CMX channel shift of <200 after desensitization protocol. The fact that patients who showed lower anti-HLA Ab levels tended to receive a transplant (see FIG. 6) must, at least in part, reflect this criteria.

Interestingly, as shown in the Examples and Figures, it was found that B cell response as analyzed by the B cell-CFC did not show a significant correlation with anti-HLA antibody levels detected in blood (FIG. 6). This indicates that B cell response as analyzed by the B cell-CFC represents different immunological status from that indicated by anti-HLA antibody levels. As such, B cell-CFC measured values better represent the actual B cell reactivity against all possible allo- and donor-Ags regardless of anti-HLA antibody levels. This may include 1) reactivity against HLA and HLA related Ags to which antibodies are currently detected in blood, 2) reactivity against HLA and HLA related Ags to which antibodies are not currently detected in blood, but were detected in the past (historic Ab positivity), 3) reactivity against other allo-Ags that are not yet identified, and 4) reactivity against Ags that are not allo-Ags, but those with which recipient B cells react or cross react to potentially cause AMR.

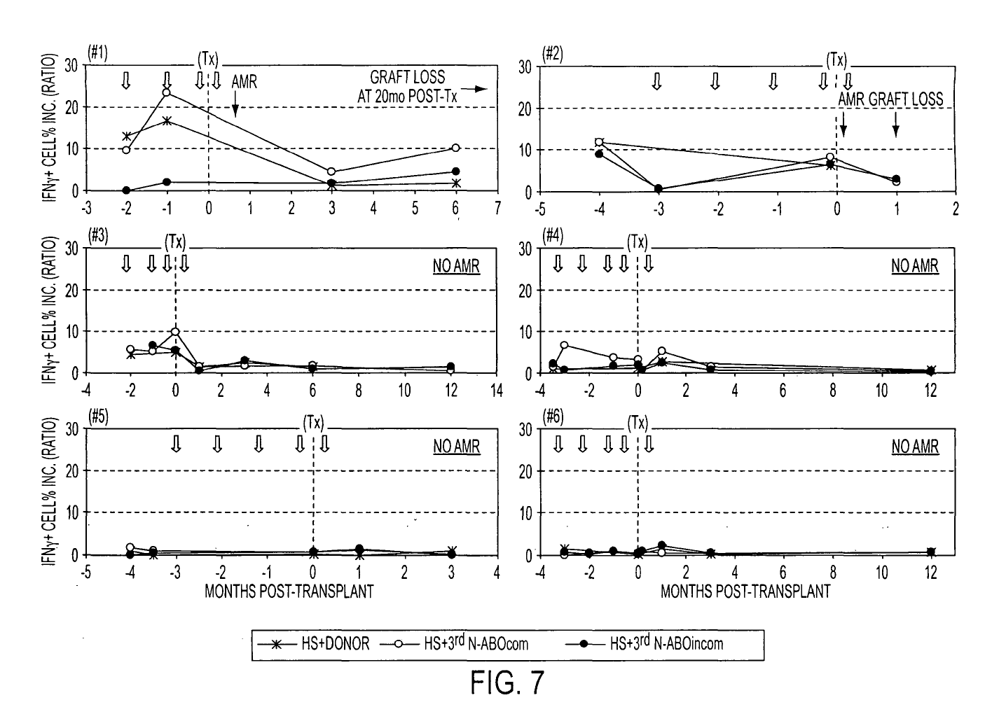

It was found that patients with high pre-Rx B cell reactivity against donor and 3^{rd} N PBMCs developed AMR, while those with no or low B cell reactivity did not (FIG. 7). Anti-HLA class I and class II antibody levels in these 6 patients were similar and were not correlated with B cell response as analyzed by the B cell-CFC. The 2 patients with AMR showed high B cell response against donor and at least one of the 3^{rd} N PBMCs at multiple time points after IVIG-Rx and before transplant (FIG. 7).

In contrast, patients without AMR showed minimal or much lower B cell response against PBMCs, especially donor PBMCs. Although patient #4 showed some B cell reactivity, those were against 3^{rd} N PBMCs and the response against their donor was minimal. B cell response in patient #3 was not minimal against donor and 3^{rd} N PBMCs, but much lower than those in the 2 patients with AMR. This level of B cell response (ratio <5×) as analyzed by B cell-CFC might be within the acceptable range for successful transplant without AMR.

Two patients with AMR received a transplant due to negative or acceptable CMX after IVIG-Rx. In retrospect, these patients may not have been ready to receive a transplant and may have required additional desensitization therapy before transplant. These results support our hypothesis that abatement of B cell activation is essential for successful transplant in this patient population and monitoring patients for B cell response using the method of the present invention can gauge IVIG-Rx efficacy and predict AMR risk post-transplant.

The results confirm that the inventive B cell-CFC assay is useful and showed significant correlation of the B cell response with sensitization status or AMR episodes, suggesting that the B cell-CFC is a useful tool for assessing the efficacy of desensitization procedures and predicting AMR post-transplant. Additional desensitization should be considered if patients show high B cell response as analyzed by the B cell-CFC, especially to donor PBMCs, even in light of an acceptable flow CMX. However, it is less clear if patients with CMX+, but negative B cell response as analyzed by the B cell-CFC, are ready for successful transplant without AMR.

There was no significant effect of IVIG-Rx on abatement of B cell activation as assessed by the B cell-CFC. B cell response against various PBMCs decreased in some patients after Rx and not in others. While not wishing to be bound by a single theory, it is believed that this is due to a majority of patients ( 9/15) being treated with low dosages of IVIG (0.5 or 1 g/kg) known to be generally ineffective for desensitization.

In conclusion, allo- and/or donor-Ag-specific B cells were elevated in many HS patients, but not in most normal individuals. B cell responses as analyzed by the B cell-CFC did not correlate with anti-HLA Ab levels, but did correlate with AMR episodes post-transplant. Thus, the inventive B cell-CFC is useful to assess the efficacy of desensitization therapy and predict AMR post-transplant.

Diagnostic Methods

The inventive assays can be used as part of a diagnostic service for determining the level of allo- and donor-Ag-specific B cells a in a patient. In some embodiments, a physician, nurse, or other medical professional draws a sample of blood from a patient. This sample may be transported to a laboratory in some embodiments for analysis. For example, blood that has been drawn the previous day may be used in this assay. This allows blood to be drawn at one location to be shipped to another for analysis within this assay.

The sample is then analyzed using the inventive assays described herein to produce a report. This report may contain an indication of the existence of allo- and/or donor specific B cells in the patient from whom blood has been drawn. The report may be in any form (e.g., paper, email, electronic document, computer display etc.) and can included, but is not limited to, allo- and/or donor-specific B cells, T-cells, and/or NK cells in a numerical or graphical form.

In some embodiments, this report is provided to a physician or other medical professional who can use its contents to evaluate the steps needed for a successful organ transplant. For example, specific compositions may be administered before or during the transplant in response to the level of allo- and/or donor specific B cells shown in the report. In some embodiments, this diagnostic method can be provided in exchange for payment.

Kits

The components of the inventive assays described herein may be packaged as kits. For example, the reagents needed for running the inventive assays may be packed in single packages or in bulk packages containing more than one set of reagents. These components may be sterile in some embodiments.

### Example 1

This research study was approved by the Institutional Review Board at Cedars-Sinai Medical Center. Before entering the study, informed consent was obtained from the patients and potential donors. Fifteen adult kidney transplant patients (male: 4, female: 11; median age 45.0, range 26-63; blood type O: 9, A: 3, B: 1, AB: 2) who were enrolled in the NIH IG03 study were tested for the B cell-CFC. These patients were desensitized between July 2003 and June 2004, and transplanted, if so, between September 2003 and September 2004 at Cedars-Sinai Medical Center. All patients entered into the NIH-IG03 study were highly-HLA sensitized and had positive complement-dependent cytotoxicity crossmatches (CDC-CMXs) with prospective living donors.

Patients meeting study criteria were treated with one of three dosages of WIG (0.5, 1 or 2 g/kg), maximum 4 doses, one month apart. CDC-CMXs against donor was tested after each IVIG infusion. If CMX results were negative or within the acceptable range (negative CDC-CMX and Flow cytometry CMX <200 channel shifts (CS) (Negative range: <50 CS for T-cells, <100 for B cells), the patient was transplanted. An additional dose of IVIG was given at day 7 post-transplant.

All transplant patients received a standard triple immunosuppressive regime consisting of calcineurin inhibitor (Cyclosporin A or tacrolimus), mycophenolate mofetil and steroid, and rabbit anti-thymocyte globulin as induction therapy. All patients received anti-CMV prophylaxis which consisted of oral Gancyclovir (o-GCV) for 4 months and oral Acyclovir (o-ACV) for 4 months for high (Donor+/Recipient−) and moderate (D+/R+, D−/R+)/low risk (D−/R−) patients, respectively. All patients except for D−/R− recipients were monitored for CMV and EBV infections by PCR monthly during the first 4 months post-transplant. Biopsies were performed after renal function declined >20% from baseline. Biopsy proven rejection episodes were treated with pulse methylprednisolone and/or rabbit anti-thymocyte globulin for cell-mediated rejection (CMR) episodes. For patients experiencing AMR (Banff Grade I and II) episodes that were C4d positive, pulse methylprednisolone (10 mg/kg/day for 3 days), WIG (2 g/kg, 1×) and Rituxan (375 mg/m², 1×) were given initially. In patients experiencing severe AMR (Banff Grade III) or thrombotic microangiopathy, treatment with plasmapheresis (3-5 sessions) followed by repeat 2 g/kg WIG and Rituxan 375 mg/m² was initiated.

Sample Collection and Processing

Heparinized whole blood samples were collected immediately before each IVIG infusion and at 1, 3, 6 and 12 months post-transplant, if patients were transplanted, and submitted for the B cell-CFC. Blood samples obtained pre-1^{st} IVIG infusion were used as the pre-IVIG-Rx time point. Blood samples were collected one month before the 1^{st} IVIG infusion and used as the pre-IVIG-Rx time point in some patients. A portion of the blood samples were centrifuged to obtain plasma samples for measurement of anti-HLA class I and class II antibody levels by ELISA.

Measurement of Allo- and Donor-Ag Specific B cell Response by Intracellular Cytokine Flow Cytometry (B cell-CFC)

The CFC assay detects intracellular cytokine produced in response to antigenic stimulation and quantifies the frequency of Ag-specific cell responses. The inventive B cell-CFC assay was performed using whole blood as previously described (29, 30). Peripheral blood mononuclear cells (PBMCs) obtained from donor or 3^{rd} party normal individuals (3^{rd} N) were used as challenge Ags.

Five tubes for different conditions (negative, positive controls, with PBMCs from donor, 3^{rd} N-ABO compatible [3^{rd} N-ABOcom], 3^{rd} N-ABO incompatible [3^{rd} N-ABOincom] to the patient) were run per sample for the assay. Ten μl of anti-CD28/49d at 1 μg/ml final concentration (BD Biosciences, San Jose, Calif.) and 10 μl of Brefeldin A (BD Biosciences) at 10 μg/ml were added to 1 ml of blood for co-stimulation and prevention of IFNγsecretion, respectively. 100 μl of this mixture was aliquoted into each of 3 tubes and 200 μl into each of 2 tubes. 100 μl of donor, 3^{rd} or ABOincom (2×10⁶/ml) irradiated PBMCs were added to each of 100 μl aliquots. Two μl of Staphylococcal Enterotoxin B (SEB, 1 μg/ml, Sigma-Aldrich, St. Louis, Mo.) were added to one of the 200 μl of aliquots as a positive control. The remaining 200 μl aliquot without additives served as a negative control.

Whole blood obtained from a 3^{rd} N was also tested for the B cell-CFC at all time points when patient blood was tested. A total of 14 3^{rd} N (Blood type O: 6, A: 6, B: 1, AB: 1) were tested and a total of 75 B cell-CFC were performed for 3^{rd} N. The conditions tested for 3^{rd} N were negative, positive controls, with PBMCs from the HS patient (ABOcom to the 3^{rd} N) tested on the same day, the donor (ABOcom to the 3^{rd} N) and 3^{rd} N-ABOincom. After 6 hours incubation at 37° C. followed by incubation at 18° C. overnight using a thermocycler, 50 μl of 20 mM EDTA were added to each tube as an additional anti-coagulant and tubes were incubated for 10 minutes to detach adherent cells.

Red blood cells were lysed in FACS Lysis solution (BD Biosciences) for 10 minutes at room temperature. Cells were washed with FACS buffer (0.5% bovine serum albumin in phosphate buffered saline [BSA-PBS] with 0.1% sodium azide) and resuspended in FACS Permeabilizing Solution 2 (BD Biosciences). After 10 minutes incubation at room temperature, cells were washed and stained with an antibody cocktail containing antibodies to CD3, CD8 (Caltag, Burlingame, Calif.) and IFNγ(BD Biosciences).

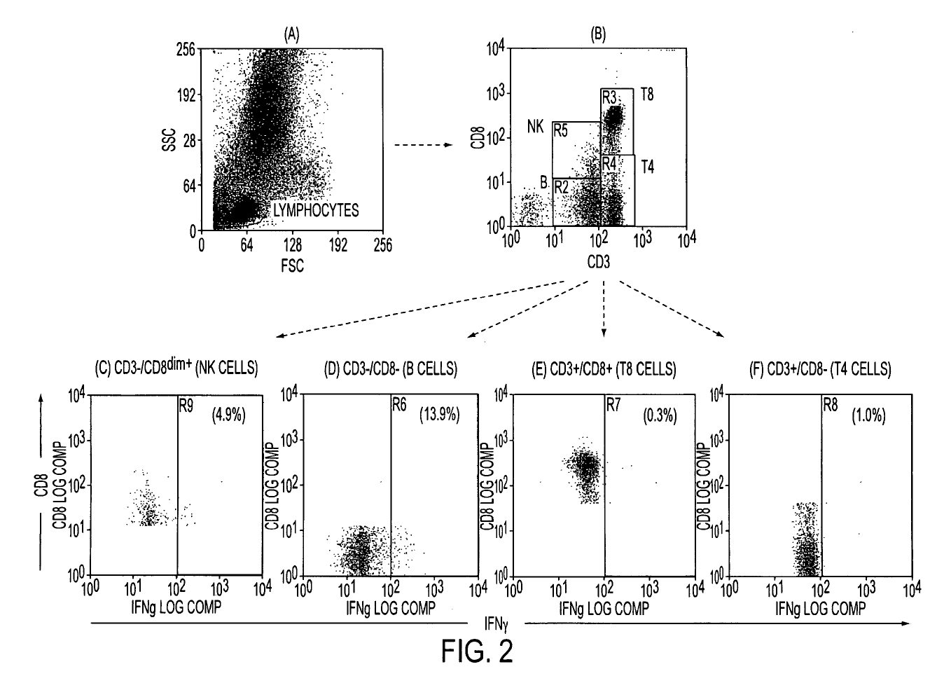

All samples were acquired within 24 hours in a flow cytometer (BD Biosciences). A total of 250,000 cells were acquired and data analysis was performed with Cellquest (BD Biosciences). Lymphocytes first gated by forward/side scatter were further plotted against CD3 and CD8 (FIG. 2). CD3+/CD8+, CD3+/CD8−, CD3−/CD8^{dim+} and CD3−/CD8− were tentatively designated as T8, T4, NK and B cells, and IFNγ+ cell % in each cell population was calculated. Response regions were defined using positive and negative controls for each sample. IFNγ+ cells were determined using visual cluster analysis (31). Results were also expressed as the ratio against IFNγ+ cell % without PBMCs or SEB stimulation in each blood sample. A ratio >1.0 represents positive response.

Allo- and Donor-Ag Preparation

PBMCs were isolated from heparinized blood obtained from donor, 3^{rd} N and/or HS patients by density-gradient centrifugation using Ficoll-Hypaque (3). PBMCs were aliquoted and frozen. Either freshly isolated or frozen PBMCs were used for the CFC. Pilot experiments showed no difference in stimulation between fresh and cryopreserved PBMCs. Immediately prior to the CFC, PBMCs were irradiated at 2500 rad.

Anti-HLA Class I and Class II Antibody ELISAs

Anti-HLA class I and class II IgG antibodies were quantified by Lambda Antigen Tray™ Mixed Class I & II ELISA kit (One Lambda, Canoga Park, Calif.) following the instruction provided by the manufacturer. Briefly, serum anti-HLA class I or class II IgG antibodies bound to mixture of affinity-purified HLA class I or class II antigens coated on an ELISA plate were detected by alkaline-phosphatase (AP)-conjugated anti-human IgG followed by adding the substrate. The OD multiplied by 1000 was used as antibody levels (units). When the OD was higher than that of the positive HLA serum control, the serum was diluted with 0.1% BSA-PBS by up to 1000 times and reanalyzed. Plasma samples obtained from heparinized blood were also used for anti-HLA class I and class II IgG antibody ELISA.

No difference in antibody levels between serum and plasma was noted in pilot experiments. Variation between assays was minimal (Positive HLA serum controls: 1435±145, Negative HLA serum controls: 120±30, Positive border line: 383±50 units in 8 ELISA plates). Anti-HLA class I and class II antibodies in 14 normal individuals (3^{rd} N) included in this study as analyzed by the ELISA were 291±253 (median 246) and 219±118 (median 183), respectively.

Statistical Analysis

Comparison of IFNg+ cell % in each cell population between pre-IVIG-Rx HS patients and 3^{rd} N was performed by non-parametric Wilcoxon rank sum test IFNγ+ cell % increase in response to various stimulators were compared to that without stimulation by a mixed models analysis.

Transplant Rate and Allograft Rejection

Fifteen patients with various blood types received IVIG-Rx with one of three dosages, one to four times as shown in Table 2.

TABLE 2
Patient demographcs and transplantation status.

| | | | Anti-HLA Abs (units)*¹ | | IVIG-Rx | | Blood Type | ABOincom |
|---|---|---|---|---|---|---|---|---|
| Patient | Age | Sex | Class I Ab | Class II Ab | (g/kg) | (# treated) | (A, B, O) | tested to*² |
| #1 | 45 | F | 732 | 1,242 | 2 | 2 | O | B |
| #2 | 39 | F | 1,702 | 94 | 2 | 4 | O | B |
| #3 | 48 | F | 740 | 198 | 1 | 2 | O | A |
| #4 | 51 | M | 102 | 98 | 1 | 3 | O | B |
| #5 | 41 | M | 209 | 212 | 2 | 4 | O | A |
| #6 | 53 | M | 136 | 103 | 0.5 | 3 | A | O |
| #7 | 52 | F | 773 | 111 | 2 | 2 | O | A |
| #8 | 40 | F | 57,600 | 101,200 | 2 | 1 (WD)*⁵ | O | A |
| #9 | 37 | M | 1,568 | 630 | 2 | 1 | A | O |
| #10 | 26 | F | 48,900 | 74,100 | 1 | 4 | AB | O |
| #11 | 34 | F | 48,950 | 984 | 1 | 4 | AB | A |
| #12 | 63 | F | 22,650 | 4,255 | 1 | 3 | A | O |
| #13 | 56 | F | 183 | 7260 | 0.5 | 3 | O | A |
| #14 | 34 | F | 9,410 | 323,600 | 0.5 | 4 | O | A |
| #15 | 46 | F | 143,900 | 4,690 | 0.5 | 2 | B | O |

| | | AMR/CMR/No AR*⁴ | | Graft Loss | |
|---|---|---|---|---|---|
| Patient | Tx/No Tx*³ | (types) | (post-Tx) | (post-Tx) | Mismatch# |
| #1 | Tx | AMR | 15 days | 1 yr 8 mo | 3 |
| #2 | Tx | AMR/CMR | 5 days | 1 mo | 5 |
| #3 | Tx | No AR | | | 6 |
| #4 | Tx | No AR | | | 1 |
| #5 | Tx | No AR | | | ? |
| #6 | Tx | No AR | | | 5 |
| #7 | No Tx | | | | |
| #8 | No Tx | | | | |
| #9 | No Tx | | | | |
| #10 | No Tx | | | | |
| #11 | No Tx | | | | |
| #12 | No Tx | | | | |
| #13 | No Tx | | | | |
| #14 | No Tx | | | | |
| #15 | No Tx | | | | |

*¹Anti-HLA Ab levels pre-IVIG-Rx
*²The type of blood used for 3^{rd} N-ABOincom PBMCs for the B cell-CFC at the pre-IVIG-Rx time point.
*³Tx-Transplant
*⁴AMR—Antibody mediated rejection, CMR—Cell mediated rejection, No AR—No allograft rejection.
*⁵This patient withdrew from the study.

Of 15 patients, 6 showed donor CMX results within the acceptable range after IVIG-Rx, and were transplanted. Of these 6, 1 patient (#1) developed AMR at 15 days post-transplant and another (#2) had AMR and CMR at 5 days post-transplant. These patients lost their grafts at 20 months and 1 month post-transplant, respectively. The remaining 4 patients without AMR and/or CMR retained their grafts during study period. Nine patients did not receive transplant because either they did not show acceptable donor CMX levels or withdrew from the study.

IFNγ+ Cell % in Pre-IVIG-Rx HS Patient Blood without PBMC Stimulation

A typical picture of the B cell-CFC result is shown in FIG. 1. Pre-IVIG-Rx blood without stimulation (base line) in HS patients showed more variance in IFNγ+ cell % compared to that in 3^{rd} N in all 4 cell populations. Although the baseline IFNγ+ cell % in HS patients and 3^{rd} N largely overlapped, the median IFNγ+ cell % in B and NK cells in HS patients were significantly lower than that in 3^{rd} N (0.23% vs. 0.69% in B cells, p=0.027, and 0.35% vs. 1.15% in NK cells, p<0.03, respectively), while T8 and T4 cells did not show a significant difference between the 2 groups (0.20% vs. 0.26% in T8 and 0.06% vs. 0.09% in T4 cells).

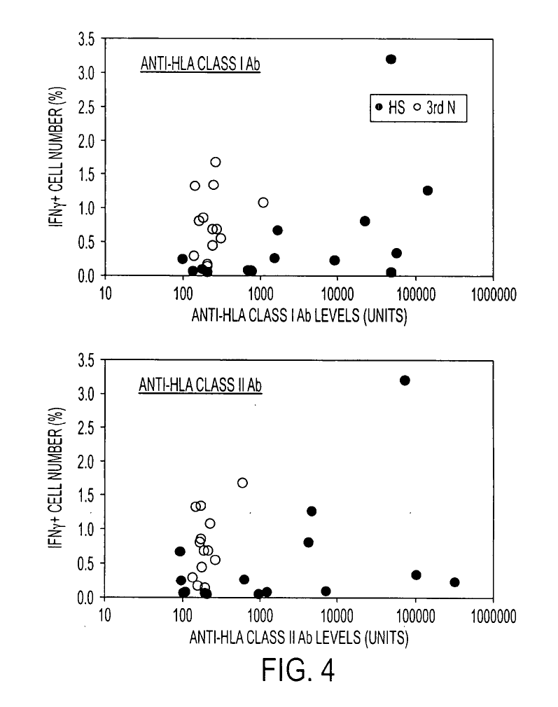

It was determined if pre-IVIG-Rx IFNγ+ B cell % in HS patients was associated with anti-HLA class I or class II antibody levels. There was no significant association between the two parameters (FIG. 6). Although higher anti-HLA class I antibody levels tended to show higher IFNγ+ B cell % in HS patients (closed circles in FIG. 4), various levels of IFNγ+ B cell % were observed regardless of the anti-HLA antibody levels. Especially when all results obtained from HS and 3^{rd} N were combined, there was no significant association between the two parameters.

IFNγ+ B Cell Increase in Response to Donor or 3^{rd} N PBMC in Pre-IVIG-Rx HS Patient

It was investigated whether IFNγ+ B cell % in pre-IVIG-Rx HS patient blood increased when blood was stimulated with allo- and/or donor-Ags expressed on PBMCs or SEB.

Many more HS patients showed positive and high response against PBMCs and SEB compared to 3^{rd} N (FIG. 7). In HS patients, the rates of IFNγ+ B cell % increase in response to PBMCs from donor, 3^{rd} or 3^{rd} N-ABOincom, or SEB were 4.5 (p=0.002), 3.0 (p=0.005), 2.4 (p=0.006) or 34.9 (p<0.001) fold against negative control without stimulation, respectively, while 3^{rd} N showed no significant increase in response to PBMCs from HS patients (1.6 fold, NS), donor (1.22 fold, NS) or 3^{rd} N-ABOincom (0.92 fold, NS). These high responses against PBMCs in HS patients were observed regardless of ABO compatibility and 3^{rd} N did not show significant positive responses against ABOincom PBMCs. The rate of increase in response to SEB in 3^{rd} N was significant (1.8 fold, p=0.005), but was significantly lower than that in HS patients (1.8 vs. 34.9 fold, p=0.036).

It was investigated whether the rate of IFNγ+ B cell % increase in response to PBMCs in pre-IVIG-Rx HS patient blood was associated with anti-HLA class I or class II antibody levels. Various rates of IFNγ+ B cell % increase in response to PBMCs were observed regardless of anti-HLA class I and class II antibody levels (FIG. 6). It should be noted that patients with lower levels of these antibodies tended to receive a transplant (diamonds in FIG. 6). Those patients transplanted with higher rates of IFNγ+ B cell % increase developed AMR (closed diamonds).

FIG. 7 shows the change of rate of IFNγ+ B cell % increase in response to various PBMCs pre-, post-IVIG-Rx and post-transplant in HS patients who received a transplant. Patients who developed AMR (#1 and #2) showed higher rates of IFNγ+ B cell % increase in response to donor and 3^{rd} N PBMCs before transplant compared to those without AMR (#3-#6). Although patient #4 showed higher response against 3^{rd} N-ABOcom after the 1^{st} IVIG infusion and 1 month post-transplant (6.8 and 5.1 fold vs. negative control without stimulation, respectively), those against donor and 3^{rd} N-ABOincom were minimal. Patient #3 also showed some response against donor and 3^{rd} N PBMCs pre-transplant. However, the responses, especially against donor, were much lower than those seen in patients with AMR.

### Example 2

Example 1 showed that in a cohort of 15 HS that allo- and DONOR-specific B cell numbers as analyzed by CFC (B cell-CFC) are elevated in many HS, but not normal controls (NC), and HS with high positive B cell-CFC may likely develop AMR. This example focuses on pre-Tx allo- and DONOR-specific-reactivity in a cohort of 55 HS patients (including the patients of Example 1) analyzed by B cell-CFC and further confirms the utility of this novel assay in the management of HS.

Methods: Blood samples obtained pre-Tx in 55 HS enrolled in the NIH IG03 study were submitted for CFC. Samples from 14 NC were also tested. Whole blood from HS or NC was incubated with irradiated peripheral blood mononuclear cells (PBMCs) (stimulator) from DONOR, 3^{rd} party with or without ABO compatibility, or from HS, DONOR or ABO incompatible 3^{rd} party, respectively. IFNγ+ cells in CD3− cells were enumerated by CFC. Results were expressed as a ratio against IFNγ+ cells without stimulation. A ratio >5 represents high positive response that can be associated with AMR.

Results: Significantly more HS had high positive B cell-CFC responses against at least one of 3 stimulators compared to NC ( 35/55 [64%] vs. 1/14 [7%], p<0.001). HS with high positive response were primarily female ( 30/41 [73%] vs. 5/14 [36%], p<0.02). 0/4 males transplanted (0%) showed high positive response, while 5/10 males not Tx (50%) did (1.5±1.1 vs. 7.3±8.5, p<0.05). No male with Tx developed AMR. Most females showed high positive response regardless of Tx status (⅝ [63%], 13.1±20.0 in Tx vs. 24/33 [74%], 9.2±10.3 in no Tx, NS). However, all 3 females with AMR showed extremely high positive response (25.1±6.2 vs. 6.2±6.2 in 5 females w/o AMR, p<0.03). The response in 5 females w/o AMR was significantly higher than that in 4 males w/o AMR (p0.03).

Conclusions: These results demonstrate the following: 1) B cell-CFC is a novel assay to measure allo- and DONOR-specific CD3− cell responses and can assess the degree of sensitization and predict AMR in HS; 2) Allo- and DONOR-specific CD3− cell numbers are elevated in many HS, but not NC; 3) Allo- and DONOR-specific-reactivity are higher in HS females; 4) HS with high positive B cell-CFC are at increased risk for AMR and may need additional pre-Tx desensitization; 5) High allo- and DONOR-specific reactivity seen in HS females may explain their higher rate of AMR; and 6) Monitoring HS using the B cell-CFC pre- and post-desensitization may help determine the efficacy of desensitization and risk for AMR.

While various embodiments of the present invention have been described above, it should be understood that they have been presented by way of example only, and not limitation. Thus, the breadth and scope of the present invention should not be limited by any of the above-described exemplary embodiments, but should instead be defined only in accordance with the following claims and their equivalents. All documents cited herein, including those in the reference list to follow, are incorporated in their entirety by reference.

### REFERENCE LIST

1. Kazatchkine M D, Kaveri S V. Immunomodulation of autoimmune and inflammatory diseases with intravenous immune globulin. N Engl J Med 2001; 345(10):747-55.
2. Dwyer J M. Manipulating the immune system with immune globulin. N Engl J Med 1992; 326(2):107-16.
3. Toyoda M, Zhang X M, Petrosian A, Wachs K, Moudgil A, Jordan S C. Inhibition of allospecific responses in the mixed lymphocyte reaction by pooled human gamma-globulin. Transpl Immunol 1994; 2(4):337-41.
4. Toyoda M, Pao A, Petrosian A, Jordan S C. Pooled human gammaglobulin modulates surface molecule expression and induces apoptosis in human B cells. Am J Transplant 2003; 3(2):156-66.
5. Tyan D B, Li V A, Czer L, Trento A, Jordan S C. Intravenous immunoglobulin suppression of HLA alloantibody in highly sensitized transplant candidates and transplantation with a histoincompatible organ. Transplantation 1994; 57(4):553-62.
6. Jordan S C, Tyan D, Stablein D, McIntosh M, Rose S, Vo A, et al. Evaluation of intravenous immunoglobulin as an agent to lower allosensitization and improve transplantation in highly sensitized adult patients with end-stage renal disease: report of the NIH IG02 trial. J Am Soc Nephrol 2004; 15(12):3256-62.
7. Jordan S C, Vo A A, Peng A, Toyoda M, Tyan D. Intravenous gammaglobulin (IVIG): a novel approach to improve transplant rates and outcomes in highly HLA-sensitized patients. Am J Transplant 2006; 6(3):459-66.
8. Glotz D, Antoine C, Julia P, Pegaz-Fiornet B, Duboust A, Boudjeltia S, et al. Intravenous immunoglobulins and transplantation for patients with anti-HLA antibodies. Transpl Int 2004; 17(1):1-8.
9. Glotz D, Antoine C, Julia P, Suberbielle-Boissel C, Boudjeltia S, Fraoui R, et al. Desensitization and subsequent kidney transplantation of patients using intravenous immunoglobulins (IVIg). Am J Transplant 2002; 2(8):758-60.
10. Jordan S C, Vo A A, Toyoda M, Tyan D, Nast C C. Post-transplant therapy with high-dose intravenous gammaglobulin: Applications to treatment of antibody-mediated rejection. Pediatr Transplant 2005; 9(2):155-61.
11. Casadei D H, del C R M, Opelz G, Golberg J C, Argento J A, Greco G, et al. A randomized and prospective study comparing treatment with high-dose intravenous immunoglobulin with monoclonal antibodies for rescue of kidney grafts with steroid-resistant rejection. Transplantation 2001; 71(1):53-8.
12. Luke P P, Scantlebury V P, Jordan M L, Vivas C A, Hakala T R, Jain A, et al. Reversal of steroid- and anti-lymphocyte antibody-resistant rejection using intravenous immunoglobulin (IVIG) in renal transplant recipients. Transplantation 2001; 72(3):419-22.
13. De Marco T, Damon L E, Colombe B, Keith F, Chatterjee K, Garovoy M R. Successful immunomodulation with intravenous gamma globulin and cyclophosphamide in an alloimmunized heart transplant recipient. J Heart Lung Transplant 1997; 16(3):360-5.
14. Montgomery R A, Zachary A A, Racusen L C, Leffell M S, King K E, Burdick J, et al. Plasmapheresis and intravenous immune globulin provides effective rescue therapy for refractory humoral rejection and allows kidneys to be successfully transplanted into cross-match-positive recipients. Transplantation 2000; 70(6):887-95.
15. Gloor J M, DeGoey S, Ploeger N, Gebel H, Bray R, Moore SB, et al. Persistence of low levels of alloantibody after desensitization in crossmatch-positive living-donor kidney transplantation. Transplantation 2004; 78(2):221-7.
16. Vieira C A, Agarwal A, Book B K, Sidner R A, Bearden C M, Gebel H M, et al. Rituximab for reduction of anti-HLA antibodies in patients awaiting renal transplantation: 1. Safety, pharmacodynamics, and pharmacokinetics. Transplantation 2004; 77(4):542-8.
17. Vo A A, Toyoda M, Peng A, Bunnapradist S, Lukovsky M, Jordan S C. Effect of induction therapy protocols on transplant outcomes in crossmatch positive renal allograft recipients desensitized with IVIG. Am J Transplant 2006; 6(10):2384-90.
18. Akalin E, Bromberg J S. Intravenous immunoglobulin induction treatment in flow cytometry cross-match-positive kidney transplant recipients. Hum Immunol 2005; 66(4):359-63.
19. Meier-Kriesche H U, Ojo A O, Hanson J A, Cibrik D M, Punch J D, Leichtman A B, et al. Increased impact of acute rejection on chronic allograft failure in recent era. Transplantation 2000; 70(7):1098-100.
20. Terasaki P I, Cai J. Humoral theory of transplantation: further evidence. Curr Opin Immunol 2005; 17(5):541-5.
21. Rifle G, Mousson C, Martin L, Guignier F, Hajji K. Donor-specific antibodies in allograft rejection: clinical and experimental data. Transplantation 2005; 79(3 Suppl):S14-8.
22. Pescovitz M D. B cells: a rational target in alloantibody-mediated solid organ transplantation rejection. Clin Transplant 2006; 20(1):48-54.
23. Martin L, Guignier F, Mousson C, Rageot D, Justrabo E, Rifle G. Detection of donor-specific anti-HLA antibodies with flow cytometry in eluates and sera from renal transplant recipients with chronic allograft nephropathy. Transplantation 2003; 76(2):395-400.
24. Takahashi K. A new concept of accommodation in ABO-incompatible kidney transplantation. Clin Transplant 2005; 19 Suppl 14:76-85.
25. Terasaki P I, Ozawa M. Predicting kidney graft failure by HLA antibodies: a prospective trial. Am J Transplant 2004; 4(3):438-43.
26. Opelz G. Non-HLA transplantation immunity revealed by lymphocytotoxic antibodies. Lancet 2005; 365(9470):1570-6.
27. Colvin R B, Smith R N. Antibody-mediated organ-allograft rejection. Nat Rev Immunol 2005; 5(10):807-17.
28. Vongwiwatana A, Tasanarong A, Hidalgo L G, Halloran P F. The role of B cells and alloantibody in the host response to human organ allografts. Immunol Rev 2003; 196:197-218.
29. Altman J D, Moss P A, Goulder P J, Barouch D H, McHeyzer-Williams M G, Bell J I, et al. Phenotypic analysis of antigen-specific T lymphocytes. Science 1996; 274(5284):94-6.
30. Radha R, Jordan S, Puliyanda D, Bunnapradist S, Petrosyan A, Amet N, et al. Cellular immune responses to cytomegalovirus in renal transplant recipients. Am J Transplant 2005; 5(1):110-7.
31. Nomura L E, Walker J M, Maecker H T. Optimization of whole blood antigen-specific cytokine assays for CD4(+) T cells. Cytometry 2000; 40(1):60-8.
32. Slifka M K, Antia R, Whitmire J K, Ahmed R. Humoral immunity due to long-lived plasma cells. Immunity 1998; 8(3):363-72.
33. Rudolphi U, Rzepka R, Batsford S, Kaufmann S H, von der Mark K, Peter H H, et al. The B cell repertoire of patients with rheumatoid arthritis. II. Increased frequencies of IgG+ and IgA+ B cells specific for mycobacterial heat-shock protein 60 or human type II collagen in synovial fluid and tissue. Arthritis Rheum 1997; 40(8):1409-19.
34. Andersson M, Yu M, Soderstrom M, Weerth S, Baig S, Solders G, et al. Multiple MAG peptides are recognized by circulating T and B lymphocytes in polyneuropathy and multiple sclerosis. Eur J Neurol 2002; 9(3):243-51.
35. Leyendeckers H, Voth E, Schicha H, Hunzelmann N, Banga P, Schmitz J. Frequent detection of thyroid peroxidase-specific IgG+ memory B cells in blood of patients with autoimmune thyroid disease. Eur J Immunol 2002; 32(10:3126-32.
36. Bernasconi N L, Traggiai E, Lanzavecchia A. Maintenance of serological memory by polyclonal activation of human memory B cells. Science 2002; 298(5601):2199-202.
37. Lanzavecchia A, Bernasconi N, Traggiai E, Ruprecht C R, Corti D, Sallusto F. Understanding and making use of human memory B cells. Immunol Rev 2006; 211:303-9.
38. Mulder A, Kardol M J, Kamp J, Uit Het Broek C, Schreuder G M, Doxiadis, I I, et al. Determination of the frequency of HLA antibody secreting B-lymphocytes in alloantigen sensitized individuals. Clin Exp Immunol 2001; 124(1):9-15.
39. Mulder A, Eijsink C, Kardol M J, Franke-van Dijk M E, van der Burg S H, Kester M, et al. Identification, isolation, and culture of HLA-A2-specific B lymphocytes using MHC class I tetramers. J Immunol 2003; 171(12):6599-603.
40. Zachary A A, Kopchaliiska D, Montgomery R A, Leffell M S. HLA-specific B cells: I. A method for their detection, quantification, and isolation using HLA tetramers. Transplantation 2007; 83(7):982-8.
41. Zachary A A, Kopchaliiska D, Montgomery R A, Melancon J K, Leffell M S. HLA-specific B cells: II. Application to transplantation. Transplantation 2007; 83(7):989-94.
42. Kuzushima K, Hoshino Y, Fujii K, Yokoyama N, Fujita M, Kiyono T, et al. Rapid determination of Epstein-Barr virus-specific CD8(+) T-cell frequencies by flow cytometry. Blood 1999; 94(9):3094-100.

What is claimed is:

1. A method for the detection of allo- or donor-specific B cells in a patient comprising:
(a) treating a sample of blood from the patient with at least one co-stimulating antibody and a Golgi body secretion inhibitor to form a treated sample;
(b) adding challenge antigens to the treated sample to form a challenged sample; and
(c) analyzing the challenged sample and a control sample to detect IFNγpositive B cells; whereby an increase in the number of IFNγpositive B cells in a challenged sample as compared to a control sample is indicative of B cell activation.

2. The method of claim 1, wherein the challenge antigen is selected from the group consisting of Peripheral Blood Mononuclear Cells, purified antigens, synthesized proteins, endothelial cells, epithelial cells, and whole blood.

3. The method of claim 1, wherein the step of analyzing uses a flow cytometer.

4. The method of claim 1, wherein the number of IFNγpositive B-cells, T cells or NK cells is analyzed.

5. The method of claim 1, wherein the antibody is selected from the group consisting of anti-CD19, anti-CD8, anti-CD4 and anti-CD16.

6. The method of claim 1, wherein the antibody is anti-ICAM 1.

7. The method of claim 1, wherein the sample is taken from the patient prior to a transplant, during a transplant, or following a transplant.

8. The method of claim 1, wherein the sample is taken before, during or after a desensitization treatment.

9. The method of claim 1, wherein the patient is a Highly Sensitized patient.

10. The method of claim 1, wherein the patient is desensitized.

11. A method of preventing antibody mediated rejection of a transplanted organ in a patient in need thereof, the method comprising:
(a) treating a sample of blood from the patient with at least one co-stimulating antibody and a Golgi body secretion inhibitor to form a treated sample;
(b) adding challenge antigens to the treated sample to form a challenged sample;
(c) analyzing the challenged sample and a control sample to detect IFNγpositive
B cells; whereby an increase in the number of IFNγpositive B cells in a challenged sample as compared to a control sample is indicative of B cell activation; and
(d) correlating the presence of allo- or donor-specific B cells with a likelihood that the patient will undergo antibody mediated graft rejection or chronic rejection.

12. The method of claim 11, further comprising identifying those patients unlikely to experience long term graft rejection as determined in (d) for a transplant procedure.

13. The method of claim 11, further comprising transplanting an organ into those patients will acceptable levels of allo- or donor-specific B cells.

14. The method of claim 11, further comprising administering desensitization therapy to those patients with unacceptable levels of allo- or donor-specific B cells.

15. The method of claim 11, further comprising administering IVIG to the patient prior to or following the organ transplant.

16. The method of claim 11, wherein the challenge antigen is selected from the group consisting of Peripheral Blood Mononuclear Cells, purified antigens, synthesized proteins, endothelial cells, epithelial cells, and whole blood.

17. The method of claim 11, wherein the step of analyzing uses a flow cytometer.

18. The method of claim 11, wherein the number of IFNγpositive B-cells, T-cells or NK cells is analyzed.

19. The method of claim 11, wherein the antibody is selected from the group consisting of anti-CD19, anti-CD8, anti-CD4 and anti-CD16.

20. The method of claim 11, wherein the antibody is anti-ICAM 1.

21. The method of claim 11, wherein the sample is taken before, during or after a desensitization treatment.

22. The method of claim 11, wherein the patient is a Highly Sensitized patient.

23. The method of claim 11, wherein the patient is administered IVIG prior to an organ transplant procedure.

24. A diagnostic method comprising:
(a) obtaining a sample of blood from a patient;
(b) treating the sample of blood with at least one co-stimulating antibody and a Golgi body secretion inhibitor to form a treated sample;
(c) adding challenge antigens to the treated sample to form a challenged sample;
(d) analyzing the challenged sample and a control sample to detect IFNγpositive B cells; whereby an increase in the number of IFNγpositive B cells in a challenged sample as compared to a control sample is indicative of B cell activation; and
(e) providing a report of the analyzed results comprising the level of allo- or donor-specific B cells in the patient.

25. The method of claim 24, wherein the challenge antigen is selected from the group consisting of Peripheral Blood Mononuclear Cells, purified antigens, synthesized proteins, endothelial cells, epithelial cells, and whole blood.

26. The method of claim 24, wherein the step of analyzing uses a flow cytometer.

27. The method of claim 24, wherein the number of IFNγpositive B-cells, T-cells or NK cells is analyzed.

28. The method of claim 24, wherein the antibody is selected from the group consisting of anti-CD19, anti-CD8, anti-CD4 and anti-CD16.

29. The method of claim 24, wherein the antibody is anti-ICAM 1.

30. The method of claim 24, wherein the sample is taken from the patient prior to a transplant, during a transplant, or following a transplant.

31. The method of claim 24, wherein the sample is taken before, during or after a desensitization treatment.

32. The method of claim 24, wherein the patient is a Highly Sensitized patient.
